(19) United States
(12) Patent Application Publication
(10) Pub. No.: US 2018/0042579 A1
(43) Pub. Date: Feb. 15, 2018

(54) ULTRASONIC DIAGNOSIS SYSTEM

(71) Applicant: Hitachi, Ltd., Tokyo (JP)

(72) Inventor: Takaya UNO, Tokyo (JP)

(73) Assignee: Hitachi, Ltd., Tokyo (JP)

(21) Appl. No.: 15/561,008

(22) PCT Filed: Mar. 23, 2016
(86) PCT No.: PCT/JP2016/059220

§ 371 Date: Sep. 22, 2017

(30) Foreign Application Priority Data
Mar. 25, 2015 (JP) 2015-062467

(51) Int. Cl. A61B 8/00; A61B 8/14

(52) U.S. Cl. CPC A61B 8/54; A61B 8/4433; A61B 8/14; A61B 8/4427; A61B 8/465; A61B 8/467; A61B 8/462

(57) ABSTRACT

Provided is an ultrasonic diagnosis system in which an ultrasonic diagnosis device is configured from a front-end (FE) device and a back-end (BE) device. The BE device is provided with two sensor pairs. Each sensor pair is configured from a light sensor and an object detection sensor. A control unit sets a backlight level adaptively on the basis of a light detection value when no object is detected. When an object is detected by either of the object sensors, the light detection value from the light detection sensor adjacent to said object is discarded.

### TECHNICAL FIELD

The present disclosure relates to an ultrasound diagnostic system, and in particular, to a technique to automatically adjust a display luminance level or the like according to an ambient light level.

### BACKGROUND

A typical ultrasound diagnostic system includes a body (cart) having a plurality of casters. An operation panel and a display are supported on the body. The operation panel has a plurality of buttons. Each button has a light emitter built therein. An ultrasound image is displayed on the display. The display is formed, for example, with a liquid crystal display (LCD). A brightness of a backlight may be varied. In addition to a cart-type ultrasound diagnostic system, ultrasound diagnostic systems formed from a plurality of devices which are independent from each other are also commercialized. In such systems, some or all of the plurality of devices are formed as a transportable device.

For example, Patent Literature 1 discloses an ultrasound diagnostic system having a first housing and a second housing. Patent Literature 2 discloses an ultrasound diagnostic system having a front-end device and a back-end device. In an ultrasound diagnostic system disclosed in Patent Literature 3, a device body and an ultrasound probe are connected wirelessly.

Normally, during ultrasound inspection, in order to observe the ultrasound image in detail, light of an inspection room is switched OFF, or the inspection room is set to a darkened state. On the other hand, in some cases, the ultrasound diagnostic system may be used in a bright general examination room, in a hospital room having a window, outdoors, or the like. Thus, it is desired to adaptively vary the brightness of the display and the brightness of each button according to an ambient light level.

Patent Literature 4 discloses an ultrasound diagnostic apparatus which can reduce or stop light emission of an operation key according to the brightness of the environment. Patent Literature 5 discloses an ultrasound diagnostic apparatus having a function to automatically adjust an ultrasound image formation condition (opacity, gain, or the like) according to a detected value of an optical sensor. Patent Literature 6 discloses an ultrasound diagnostic apparatus having a function to automatically adjust a display condition according to the ambient light level.

### CITATION LIST

### Patent Literature

Patent Literature 1: JP 2011-5241 A

Patent Literature 2: JP 2008-114065 A

Patent Literature 3: JP 2011-87841 A

Patent Literature 4: JP 2006-305303 A

Patent Literature 5: JP 2006-20777 A

Patent Literature 6: JP 2008-272473 A

### SUMMARY

### Technical Problem

In an ultrasound diagnostic system, when a display luminance level or the like is to be adaptively set according to the ambient light level, an optical detector that detects the ambient light level is placed at some position on the ultrasound diagnostic system. When the optical detector is covered by a hand, a head, or the like of the inspector, the ambient light level cannot be accurately detected. In this case, the control of the display luminance level or the like becomes inappropriate.

For example, a configuration may be considered in which a tablet terminal, in which a display and an inputter are integrated, is used as a whole or a part of an ultrasound diagnostic system, and an optical detector is provided on the tablet terminal. In this case, when the user extends a hand to an upper part of the display surface for operation, the optical detector may be covered with the hand. When the input is to be done by a gesture or a hand motion, the hand is moved in a space in front of the display surface. In this case also, the optical detector may be covered by the hand. When a tablet terminal is used, a frame portion thereof is held with the hand. In this case, the optical detector may be hidden by the hand. In addition to these cases, when the inspector moves close to the tablet terminal and attempts to look into the tablet terminal, a shadow of the head may be cast on the optical detector. It is desired that the display luminance level or the like is not unnecessarily changed due to influences of these disturbances.

An advantage of the present disclosure lies in that, in an ultrasound diagnostic system, influence of disturbances are reduced when a display condition such as a display luminance level is adaptively set according to the ambient light level. An alternative advantage of the present disclosure lies in that, in an ultrasound diagnostic system, when a tablet terminal is used, visibility of the tablet terminal is improved without reducing operability of the tablet terminal.

### Solution to Problem

According to one aspect of the present disclosure, there is provided an ultrasound diagnostic system comprising: a display that displays an ultrasound image which is formed based on a reception signal obtained by transmission and reception of ultrasound; a first optical detector that detects an ambient light; a first object detector that has an object detection field-of-view which is in a relationship of entirely or partially overlapping a light detection field-of-view of the first optical detector; and a control unit that adaptively sets a luminance level of the display based on a first light detection value of the first optical detector when no object is detected by the first object detector.

In the above-described configuration, the first object detector is provided with the first optical detector. The light detection field-of-view of the first optical detector and the object detection field-of-view of the first object detector entirely or partially overlap each other. Here, each field of view refers to a three-dimensional directive range (detection region). Therefore, for example, it becomes possible to detect a state where the first optical detector is covered by the hand of the operator (occurrence of disturbance) using the first object detector. By increasing a degree of match (degree of overlap) between the light detection field-of-view and the object detection field-of-view, a detection probability of occurrence of disturbance can be increased (at the same time, probability of erroneous detection or no detection can be reduced). When an object is detected by the first object detector, the control unit discards the first light detection value, and thus, in this case, the first light detection value is not referred to in the luminance level control. When the first object detector does not detect an object, it is highly likely that the first light detection value is a correct value which is not affected by a disturbance, the adaptive setting of the luminance level based on the first light detection value is executed. Alternatively, in place of or in addition to the luminance level control, an amount of light emission of the button or the like may be adaptively varied. Alternatively, in place of judging presence or absence of an object based on the detection value of the first object detector, an existence probability (degree) of an object may be judged and control may be executed based thereon.

According to another aspect of the present disclosure, the first optical detector and the first object detector form a first detector pair, a second detector pair is provided including a second optical detector that detects the ambient light, and a second object detector that has an object detection field-of-view which is in a relationship of entirely or partially overlapping a light detection field-of-view of the second optical detector, and the control unit adaptively sets the luminance level based on the first light detection value of the first optical detector when no object is detected by the first object detector, and a second light detection value of the second optical detector when no object is detected by the second object detector. By providing a plurality of detector pairs, it becomes possible to increase the probability that a light detection value which is not affected by the disturbance can be acquired when disturbance occurs partially or locally. Further, by referring to the plurality of light detection values even when there is no disturbance, it is possible to more accurately recognize the ambient light level. This is because the ambient light level which is detected varies depending on an orientation and a position of each sensor. For example, the luminance level may be determined based on an average value of a plurality of light detection values. For the adaptive variation of the luminance level, it is desirable to enable adjustment of the responsiveness.

According to another aspect of the present disclosure, the control unit discards the first light detection value when an object is detected by the first object detector, and discards the second light detection value when an object is detected by the second object detector. With such a configuration, it becomes possible to determine the luminance level based on light detection values which are not affected by the disturbance. For example, when it is judged that both of two light detection values are affected by the disturbance, both light detection values are discarded. In this case, the luminance level may be determined based on one or a plurality of detection values in the past, or the luminance level at that point may be maintained.

According to another aspect of the present disclosure, the first detector pair and the second detector pair are placed with orientations that are different from each other. With such a configuration, a plurality of light detection values can be obtained under a plurality of light detection conditions that are different from each other, and thus, it is possible to identify a more general ambient light level without depending too much on the directivity and placement position of the detectors.

According to another aspect of the present disclosure, the ultrasound diagnostic system comprises a front-end device having a transmission and reception circuit, and a back-end device serving as a tablet terminal having the display, the back-end device has a frame member provided around a display screen of the display, and the first detector pair and the second detector pair are provided on the frame member. According to another aspect of the present disclosure, the first detector pair is provided at or near a top right corner portion of the frame member, and facing a front side, and the second detector pair is provided at or near the top right corner portion of the frame member, facing a direction other than the front side. In the ultrasound diagnosis, the portion of the top right corner or a nearby region of the frame member is a portion where the probability of being held by the hand is relatively low. Therefore, by providing the two detector pairs there, it is possible to reduce the probability of the detector pairs being hidden by the hand. At the same time, the probability of a shadow of the hand, head, or the like being cast on the detector pairs can be reduced.

According to another aspect of the present disclosure, the first object detector is a motion detector that detects motion of a hand of an inspector, and the control unit discards the first light detection value of the first optical detector when the motion of the hand is detected by the first object detector. When a gesture input is executed to operate the screen by motion of the hand without contact in a space in front of the display screen, the probability of the shadow of the hand being cast on each optical detector becomes high. Therefore, it is desirable that the light detection value is not referred to during the gesture input, and the light detection value is referred to in a non-gesture input state in which no such input is present.

According to another aspect of the present disclosure, there is provided a back-end device serving as a tablet terminal forming a part of an ultrasound diagnostic system, the back-end device comprising: a first optical detector that detects an ambient light; a first object detector that has an object detection field-of-view which is in a relationship of entirely or partially overlapping a light detection field-of-view of the first optical detector; a second optical detector that detects the ambient light; a second object detector that has an object detection field-of view which is in a relationship of entirely or partially overlapping a light detection field-of-view of the second optical detector; and a control unit that adaptively sets a display condition of a display based on a first light detection value of the first optical detector when no object is detected by the first object detector, and a second light detection value of the second optical detector when no object is detected by the second object detector.

In the above-described configuration, the back-end device acts together with or cooperates with a front-end device, to execute ultrasound diagnosis. The above-described control is desirably executed when the back-end device is in a separated state, and also when the back-end device is in a docked state. Alternatively, a judgment condition or the like may be set differently between the separated state and the docked state.

### Advantageous Effects of Invention

According to various aspects of the present disclosure, in an ultrasound diagnostic system, when a display condition such as a display luminance level is adaptively varied according to the ambient light level, the influence by the disturbance can be reduced. Alternatively, in an ultrasound diagnostic system, when a tablet terminal is used, visibility of the tablet terminal can be improved without reducing operability of the tablet terminal.

### BRIEF DESCRIPTION OF DRAWINGS

FIG. 1 is a conceptual diagram showing an ultrasound diagnostic system according to an embodiment of the present disclosure.

FIG. 2 is a perspective diagram of an ultrasound diagnostic system in a separated state.

FIG. 3 is a perspective diagram of an ultrasound diagnostic system in a docked state.

FIG. 4 is a block diagram of a front-end device.

FIG. 5 is a block diagram of a back-end device.

FIG. 6 is a diagram showing a communication scheme in a docked state and a communication scheme in a separated state.

FIG. 7 is a diagram showing a first example placement of two sensor pairs.

FIG. 8 is a diagram showing a second example placement of two sensor pairs.

FIG. 9 is a diagram showing a third example placement of two sensor pairs.

FIG. 10 is a diagram showing an example structure for adaptive control of a backlight value.

FIG. 11 is a diagram showing a first example control by the controller shown in FIG. 10.

FIG. 12 is a diagram showing a second example control by the controller shown in FIG. 10.

### DESCRIPTION OF EMBODIMENTS

An embodiment of the present disclosure will now be described with reference to the drawings.

### (1) Ultrasound Diagnostic System

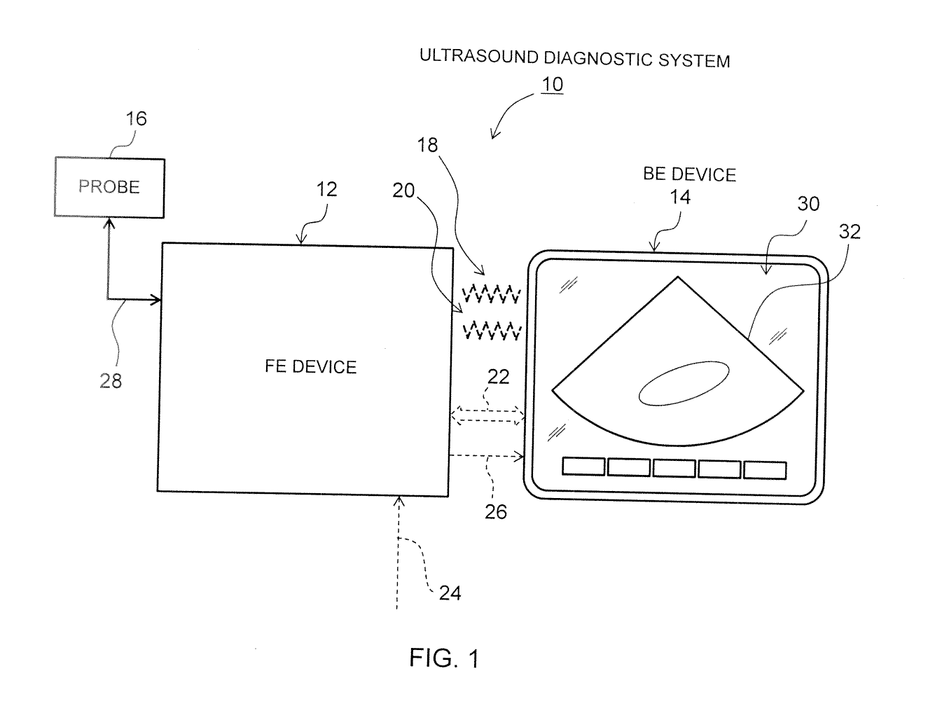

FIG. 1 schematically shows a structure of an ultrasound diagnostic system according to an embodiment of the present disclosure. An ultrasound diagnostic system 10 is a medical device used in a medical institution such as hospitals, and executes ultrasound diagnosis on a subject (living body). The ultrasound diagnostic system 10 is roughly formed from a front-end (FE) device 12, a back-end (BE) device 14, and a probe 16. The FE device 12 is a device closer to the living body, and the BE device 14 is a device which is farther away from the living body. The FE device 12 and the BE device 14 are provided as separate structures, and each forms a transportable device. The FE device 12 and the BE device 14 can be operated in a separated state in which the devices are detached from each other, and can also be operated in a docked state where the devices are put together. FIG. 1 shows the separated state.

The probe 16 is a transmitting and receiving device which transmits and receives ultrasound in a state where the probe 16 contacts a surface of the living body. The probe 16 comprises a 1-D array transducer having a plurality of transducer elements arranged in a liner shape or an arc shape. An ultrasound beam is formed by the array transducer, and is repeatedly electrically scanned. For each electric scan, a beam scan plane is formed in the living body. As the electric scanning scheme, an electric linear scan scheme, an electric sector scan scheme, and the like are known. Alternatively, a 2-D array transducer which can form a three-dimensional echo data capturing space may be provided in place of the 1-D array transducer. In the example configuration of FIG. 1, the probe 16 is connected to the FE device 12 via a cable 28. The probe 16 may alternatively be connected to the FE device 12 by wireless communication. In this case, a wireless probe is used. Alternatively, in a state where a plurality of probes are connected to the FE device 12, a probe 16 to be actually used may be selected from among the connected probes. Alternatively, a probe 16 which is to be inserted into a body cavity may be connected to the FE device 12.

In the separated state shown in FIG. 1, the FE device 12 and the BE device 14 are electrically connected to each other by a wireless communication scheme. In the present embodiment, the devices are connected to each other by a first wireless communication scheme and a second wireless communication scheme. In FIG. 1, a wireless communication path 18 using the first wireless communication scheme and a wireless communication path 20 using the second wireless communication scheme are explicitly shown. The first wireless communication scheme is faster compared to the second wireless communication scheme, and in the present embodiment, ultrasound reception data is transferred from the FE device 12 to the BE device 14 using the first wireless communication scheme. That is, the first wireless communication scheme is used for data transfer. The second wireless communication scheme is a communication scheme which is slower and simpler than the first wireless transfer scheme, and in the present embodiment, control signals are transferred from the BE device 14 to the FE device 12 using the second wireless communication scheme. In other words, the second wireless communication scheme is used for control.

In the docked state in which the FE device 12 and the BE device 12 are physically combined, the FE device 12 and the BE device 14 are electrically connected to each other by a wired communication scheme. Compared to the above-described two wireless communication schemes, the wired communication scheme is significantly faster. In FIG. 1, a wired communication path 22 between the two devices is shown. A power supply path 26 is for supplying direct current electric power from the FE device 12 to the BE device 14 in the docked state. The electric power is used for the operations of the BE device 14, and is also used for charging a battery in the BE device 14.

Reference numeral 24 represents a DC power supply line supplied from an AC adaptor (AC/DC converter). The AC adapter is connected to the FE device 12 as necessary. The FE device 12 also has a battery built therein, and can operate using the battery as a power supply. The FE device 12 has a box-like form, as will be described later. Structure and operation of the FE device 12 will be described later in detail.

Meanwhile, in the present embodiment, the BE device 14 has a tablet form or a flat plate form. The BE device 14 thus has a basic structure similar to a typical tablet computer. The BE device 14 is equipped with various dedicated software for ultrasound diagnosis, including an operation control program, an image processing program, or the like. The BE device 14 has a display panel 30 with a touch sensor. The display panel 30 functions as a user interface having functions of both an inputter and a display. In FIG. 1, a B-mode tomographic image is displayed on the display panel 30 as an ultrasound image. The user executes various inputs using a group of icons displayed on the display panel 30. It is also possible to execute a slide operation, an enlarging operation, or the like on the display panel 30.

Depending on the diagnosis usage and preferences of the inspector, or the like, the ultrasound diagnosis system 10 can be operated in a usage form selected from the separated state and the docked state. Thus, an ultrasound diagnostic system can be provided having a superior usability.

In order to prevent the operations of the ultrasound diagnostic system 10 becoming unstable or inappropriate during a state change, in the present embodiment, during the state change, control is executed to forcefully put the ultrasound diagnostic system 10 into a freeze state. Specifically, in the process of transferring from the separated state to the docked state, the FE device 12 and the BE device 14 each judge a time immediately before the docking, based on a radio wave intensity or reception state indicating a distance between the FE device 12 and the BE device 14, and control is executed in each of the devices 12 and 14 to transfer the operation state to the freeze state according to the judgment. After the docked state is established and an operation to release freeze by the inspector, the freeze states of the devices 12 and 14 are released. In the process of transfer from the docked state to the separated state, the FE device 12 and the BE device 14 individually detect that the devices are set in the separated state by line detection or other methods, and the devices are set in the freeze state. Then, after an operation to release the freeze, the freeze states of the devices 12 and 14 are released.

The BE device 14 may be separately connected to an in-hospital LAN via a wireless communication scheme or a wired communication scheme. The communication paths for these communications are not shown in FIG. 1. Alternatively, the BE device 14 (or the FE device 12) may be separately connected to other dedicated devices (for example, a remote controller) which functions for the ultrasound diagnosis, by a wireless communication scheme or a wired communication scheme.

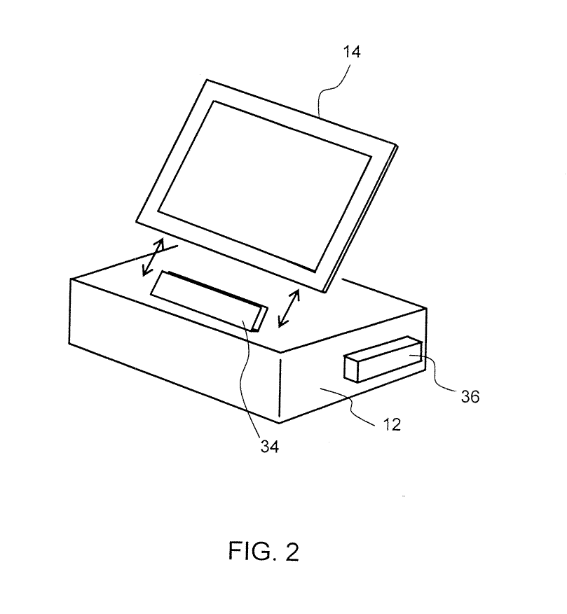

FIG. 2 shows a separated state. The FE device 12 is placed, for example, on a desk. The FE device 12 has a holder 34 having an insertion port (slot). The holder 34 has a hinge mechanism, and can rotate about a horizontal axis. A connector provided on an end of a probe cable is attached on a given side surface of the FE device 12. Alternatively, a compartment for housing the probe or the like may be formed inside the FE device 12. Such a configuration is convenient when the ultrasound diagnostic system is transported, and can protect the probe. In FIG. 2, the BE device 14 is separated from the FE device 12, and the BE device 14 can be moved farther away from the FE device 12, so long as the wireless communication is possible.

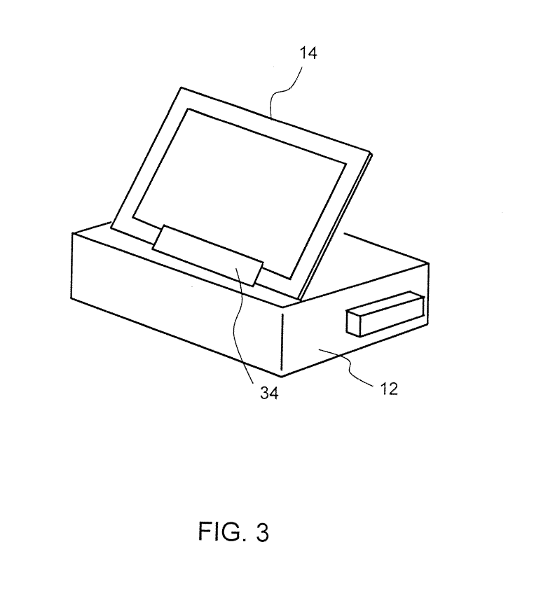

FIG. 3 shows a docked state. A lower end of the BE device 14 is inserted into the insertion port of the holder 34. In this inserted state, the FE device 12 and the BE device 14 are in a wired connection state. In other words, the devices are connected by a wired LAN, and are connected by a wired power supply line. In the docked state, an angle of the BE device 14 can be arbitrarily varied, to change the orientation thereof. BE device 14 may be tilted completely to the back surface side thereof (upper surface side of the FE device 12).

### (2) Front-End Device

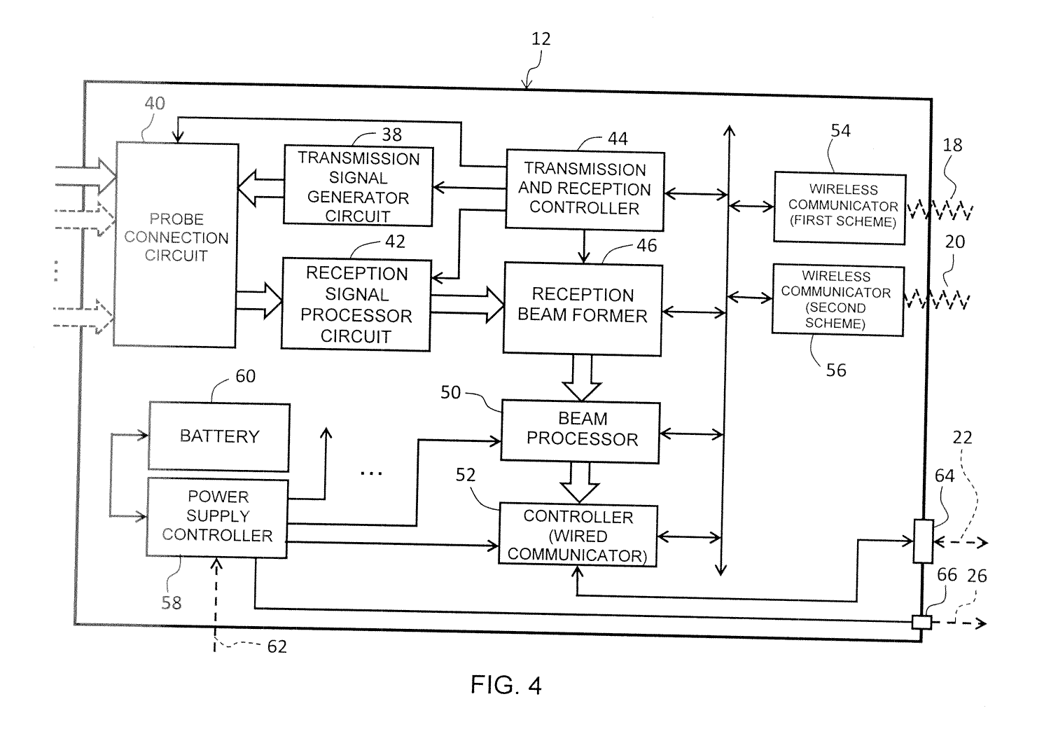

FIG. 4 is a block diagram of the FE device 12. Each block in FIG. 4 is formed with hardware such as a processor, an electronic circuit, or the like. A transmission signal generator circuit 38 is a circuit which supplies a plurality of transmission signals in parallel to each other to the plurality of transducer elements in the probe via a probe connection circuit 40. With the supply of the signals, a transmission beam is formed at the probe. When a reflection wave from within the living body is received by the plurality of transducer elements, a plurality of reception signals are output from the transducer elements, and are input to a reception signal processor circuit 42 via the probe connection circuit 40. The reception signal processor circuit 42 includes a plurality of pre-amplifiers, a plurality of amplifiers, and a plurality of A/D converters, and the like. A plurality of digital reception signals which are output from the reception signal processor circuit 42 are sent to a reception beam former 46. The reception beam former 46 applies phasing addition on the plurality of digital reception signals, and outputs beam data as a signal after the phasing addition. The beam data comprises a plurality of echo data corresponding to the reception beams and arranged in a depth direction. Reception frame data is formed by a plurality of beam data obtained by one electric scan.

A transmission and reception controller 44 controls the transmission signal generation and the reception signal process based on transmission and reception control data sent from the BE device. A beam processor 50 is a circuit which applies various data processes such as wave detection, logarithmic conversion, correlation, or the like on individual beam data which are input in a time sequential order. A control unit 52 controls an overall operation of the FE device 12. In addition, the control unit 52 executes control for wired transfer or wireless transfer, to the BE device, of beam data which are sequentially sent from the beam processor 50. In the present embodiment, the control unit 52 also functions as a wired communicator. A wireless communicator 54 is a module for executing communication in the first wireless communication scheme. A wireless communicator 56 is a module for executing communication in the second wireless communication scheme. Reference numeral 18 shows the wireless communication path according to the first wireless communication scheme, and reference numeral 20 shows the wireless communication path according to the second wireless communication scheme. The paths are bidirectional transfer paths, but in the present embodiment, a large amount of reception data is transferred from the FE device 12 to the BE device using the former, and control signals are transferred from the BE device to the FE device 12 using the latter. Reference numeral 64 shows a wired communication terminal, and the wired communication path 22 is connected thereto. Reference numeral 66 shows a power supply terminal, and the power supply line 26 is connected thereto. As described above, the power supply line 26 is a line for supplying the direct current electric power from the FE device 12 to the BE device.

A battery 60 is, for example, a lithium ion type battery, and charging and discharging therein are controlled by a power supply controller 58. At the time of battery drive, the electric power from the battery 60 is supplied via the power supply controller 58 to the circuits in the FE device 12. Reference numeral 62 shows a power supply line at the time when the AC adaptor is connected. At the time when the AC adaptor is connected, external electric power is supplied to the circuits in the FE device 12 by an action of the power supply controller 58. In this process, if the charge of the battery 60 is less than 100%, the battery 60 is charged using the external electric power.

During an ultrasound diagnosis operation (during transmission and reception), the FE device 12 repeatedly executes supply of a plurality of transmission signals to the probe and processing of a plurality of reception signals obtained thereafter, according to the control from the side of the BE device. The time sequential beam data obtained as a result of this process is consecutively transferred to the BE device by the wireless communication in the separated state and by the wired communication in the docked state. In this process, individual beam data is converted into a plurality of packets, and individual beam data is transferred by a so-called packet transfer scheme.

As the operation mode, in addition to the B mode, there are known various modes such as a CFM mode, an M mode, and a D mode (a PW mode and a CW mode). Alternatively, transmission and reception processes for harmonic imaging and elasticity information imaging may be executed. In FIG. 1, circuits such as a living body signal input circuit are not shown.

### (3) Back-End Device

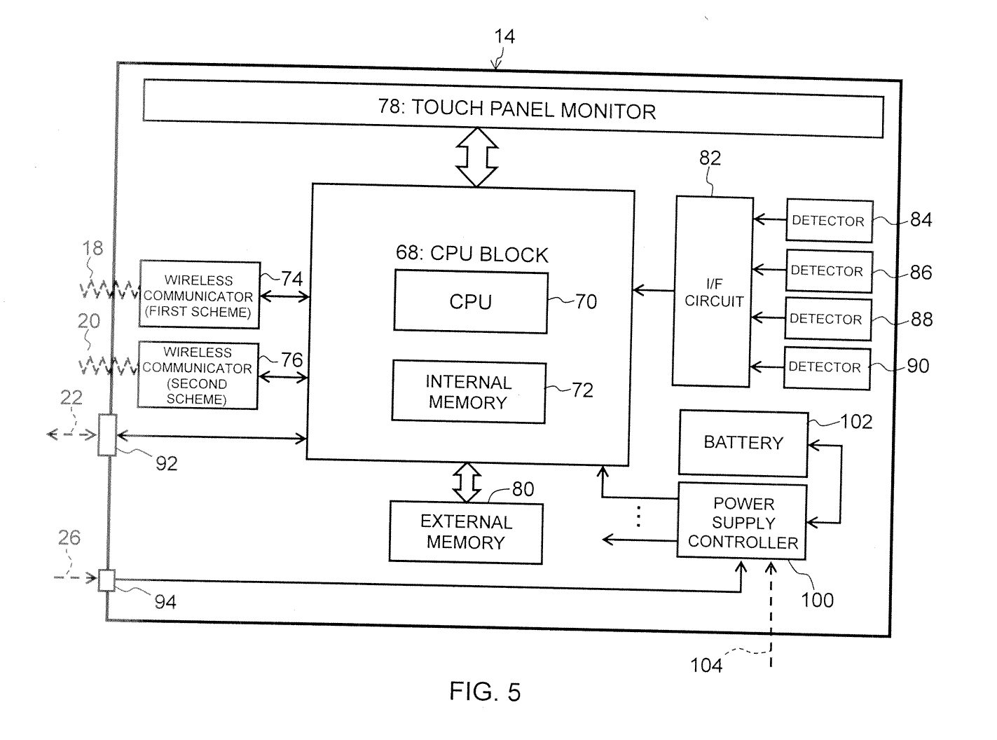

FIG. 5 is a block diagram of the BE device 14. In FIG. 5, each block shows hardware such as a processor circuit, a memory, or the like. A CPU block 68 comprises a CPU 70, an internal memory 72, or the like. The internal memory 72 functions as a working memory or a cache memory. An external memory 80 connected to the CPU block 68 stores an OS, various control programs, various processing programs, or the like. The latter includes a scan convert process program. The external memory 80 also functions as a cine-memory having a ring buffer structure. The cine-memory may alternatively be formed in the internal memory 72.

The CPU block 68 generates display frame data by a scan convert process based on a plurality of beam data. The display frame data forms an ultrasound image (for example, a tomographic image). The process is consecutively executed, to generate a video image. The CPU block 68 applies various processes on the beam data or the image for ultrasound image display. In addition, the CPU block 68 controls the operation of the BE device 14, and controls the ultrasound diagnostic system as a whole.

A touch panel monitor (display panel) 78 functions as an input device and a display device. Specifically, the touch panel monitor 78 includes a liquid crystal display and a touch sensor, and functions as a user interface. On the touch panel monitor 78, a display image including the ultrasound image is displayed, and various buttons (icons) for operation are displayed.

A wireless communicator 74 is a module for the wireless communication according to the first wireless communication scheme. A wireless communication path for this wireless communication is shown with reference numeral 18. A wireless communicator 76 is a module for the wireless communication according to the second wireless communication scheme. A wireless communication path for this wireless communication is shown with reference numeral 20. The CPU block 68 also has a function to execute a wired communication according to a wired communication scheme. In the docked state, a wired communication line is connected to a wired communication terminal 92. In addition, the power supply line 26 is connected to a power supply terminal 94.

A plurality of detectors 84˜90 are connected to the CPU block 68 via an I/F circuit 82. The detectors include an illumination sensor (optical detector), a proximity sensor (object detector), a temperature sensor, or the like. Alternatively, a module such as a GPS may be connected. The I/F circuit 82 functions as a sensor controller.

A battery 102 is a lithium-ceramic type battery, and charging and discharging thereof are controlled by a power supply controller 100. The power supply controller 100 supplies the electric power from the battery 102 to the circuits in the BE device 14 during a battery operation. During non-operation of the battery, the electric power supplied from the FE device or the electric power supplied from the AC adaptor is supplied to the circuits in the BE device 14. Reference numeral 104 shows a power supply line via the AC adaptor.

The BE device 14 controls the FE device, sequentially processes beam data sent from the FE device to generate an ultrasound image, and displays the ultrasound image on the touch panel monitor 78. In this process, an operation graphic image is also displayed with the ultrasound image. In a normal real-time operation, the BE device 14 and the FE device are electrically connected to each other wirelessly or with a wire, and, while the devices are synchronized, the ultrasound diagnosis operation is continuously executed. In the freeze state, in the BE device 14, operations of the transmission signal generator circuit and the reception signal generator circuit are stopped, and operation of a voltage boosting circuit in the power supply controller 100 is also stopped. In the BE device, a static image display is set at the time of freeze, and the content is maintained. Alternatively, the BE device may be configured such that an external display may be connected to the BE device.

### (4) Communication Schemes

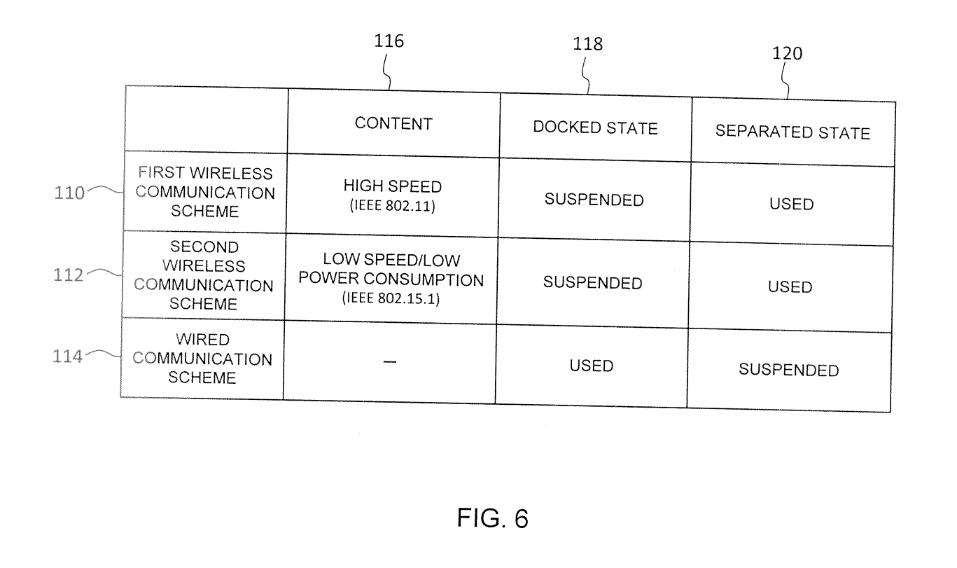

FIG. 6 collectively shows communication schemes used in a docked state 118 and a separated state 120. Reference numeral 110 shows the first wireless communication scheme, and reference numeral 112 shows the second wireless communication scheme. Reference numeral 114 shows the wired communication scheme. Reference numeral 116 shows contents of the wireless communication scheme. In the docked state 118, the wired communication is selected, and the first wireless communicator and the second wireless communicator are set in an operation suspended state in the FE device and the BE device. With such a configuration, electric power can be saved. On the other hand, in the separated state 120, the wireless communication is selected, and the first wireless communicator and the second wireless communicator operate in the FE device and the BE device. In this process, the wireless communication system is set in an operation suspended state. The first wireless communication scheme 110 is faster than the second wireless communication scheme 112. In other words, the second wireless communication scheme 112 is slower than the first wireless communication scheme 110, but is simpler and less expensive, and consumes less power. As the wired communication scheme, a TCP/IP protocol over Ethernet (Registered Trademark) may be exemplified. As the first wireless communication scheme, IEEE 802.11 may be exemplified, and, as the second wireless communication scheme, IEEE 802.15.1 may be exemplified. These are merely exemplary, and other communication schemes may be used. In any event, a secure communication scheme is desirably used.

In the present embodiment, the wireless communicator according to the second wireless communication scheme 112 has a function to automatically vary a transmission power according to a reception intensity (that is, distance). In other words, when the BE device comes near the FE device, control to reduce the transmission power is automatically executed in both devices. Therefore, it is possible to judge that the devices have moved close to each other based on the set transmission power. Alternatively, it is also possible to judge that the two devices have moved close to each other based on the reception intensity, a reception error rate, or the like, in place of the transmission power. Further alternatively, a proximity sensor may be used.

### (5) Adaptive Adjustment of Display Luminance Level at the Back-End Device

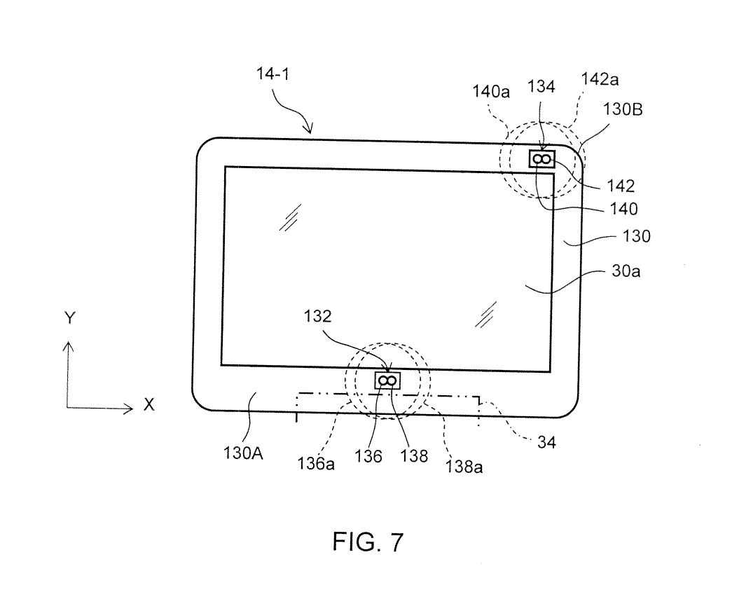

FIG. 7 shows a first example placement of two sensor pairs. A BE device 14-1 corresponds to the BE device 14 shown in FIG. 1 or the like. That is, the BE device 14-1 forms a tablet terminal. The BE device 14-1 has a display screen 30a. The display screen 30a forms a display and also an inputter. Specifically, as shown in FIG. 5, the BE device 14-1 has a touch panel monitor. A periphery around the display screen 30a, that is, a region outside of the display screen 30a, is a frame member 130. The frame member 130 forms a bezel. In FIG. 7, an X direction and a Y direction which are in an orthogonal relationship are defined. The display screen 30a is parallel to a plane defined by the X direction and the Y direction. A direction orthogonal to the X direction and the Y direction is a Z direction, and is a direction through the display screen 30a. The X direction is a left-and-right direction, and the Y direction is an up-and-down direction.

At a lower part of the frame member 130, that is, on a lower side 130A of the display screen 30a, a first sensor pair 132 is embedded. On the other hand, at or near a top right corner portion of the frame member 130, a second sensor pair 134 is embedded. The first sensor pair 132 comprises a first optical sensor 136 and a first object sensor 138. Reference numeral 136a indicates a detection field-of-view of the first optical sensor 136, and reference numeral 138a indicates a detection field-of-view of the first object sensor 138. In FIG. 7, the forms of the detection fields-of-view are merely exemplary.

The detection fields-of-view 136a and 138a, which are in a paired relationship, substantially overlap each other. The first optical sensor 136 and the first object sensor 138 are provided such that the fields of view 136a and 138a at least partially overlap each other. In FIG. 7, two circles indicated as the fields of view 136a and 138a show the width of the respective field of view in the XY plane. The sizes of the circles, however, are merely exemplary.

The first sensor pair 132 is provided in such a manner that, when the BE device 14 and the FE device are in the docked state, the sensor pair is not hidden by the holder 34 (refer to FIGS. 2 and 3). That is, the first sensor pair 132 is placed on the BE device 14-1 so that the first sensor pair 132 is exposed more to an upper side than an upper side of the holder 34 in the docked state. In the example configuration of FIG. 7, the first sensor pair 132 is provided at a center in the X direction in the lower side 130A. Alternatively, the first sensor pair 132 may be provided on other positions, so long as the functions of the first sensor pair 132 can be realized.

The detection fields-of-view 136a and 138a may have a substantial hemispherical form. Alternatively, the fields of view may have a directivity which is more three-dimensionally narrowed. Further, the detection field-of-view may be a field of view covering the entirety of a front side of the display screen 30a.

The first optical sensor 136 is a sensor which detects an ambient light level. For example, an illumination sensor may be used as such a sensor. The first object sensor 138 is a sensor which detects presence/absence of an object in the detection field-of-view 138a. As such a sensor, an optical sensor, an ultrasound sensor, or the like may be used. Alternatively, as the object sensor 138a, a motion sensor or a gesture sensor may be provided. In this case, a two-dimensional sensor such as a CCD may be provided. In the present embodiment, two sensors 136 and 138 of the first sensor pair 132 are placed in proximity to each other. Alternatively, the two sensors 136 and 138 may be provided distanced from each other. Desirably, positions and orientations of the sensors are set such that the detection fields-of-view of the two sensors of the sensor pair substantially overlap each other as described above. In the example configuration of FIG. 7, the two sensors 136 and 138 face in the Z direction. In other words, the central axes thereof face the front side.

In the first example placement of FIG. 7, in addition to the first sensor pair 132, a second sensor pair 134 is provided. As described above, the second sensor pair 134 is provided at or near the top right corner portion of the frame member 130.

The second sensor pair 134 includes a second optical sensor 140 and a second object sensor 142. The sensors 140 and 142 are placed in proximity to each other, and detection fields-of-view thereof are shown with reference numerals 140a and 142a, respectively. The detection fields-of-view 140a and 142a shown in the figure are merely for convenience, and various sizes may be conceived for the fields of view.

In the example configuration of FIG. 7, the second sensor pair 134 basically has the same structure and function as the first sensor pair 132. The first sensor pair 132 and the second sensor pair 134 only differ from each other in the placement positions. Therefore, the sensors 140 and 142 will not be described here in detail. When the first sensor pair 132 and the second sensor pair 134 are placed on the frame member 130, the sensor pairs are placed while avoiding various switches, light emitters, or the like provided separately on the frame member. Because each of the sensor pairs 132 and 134 has a small size, a thickness and a width of the frame member 130 are not increased by the placement of the sensor pairs. The second sensor pair 134 may be placed at other positions so long as the functions of the second sensor pair 134 can be realized.

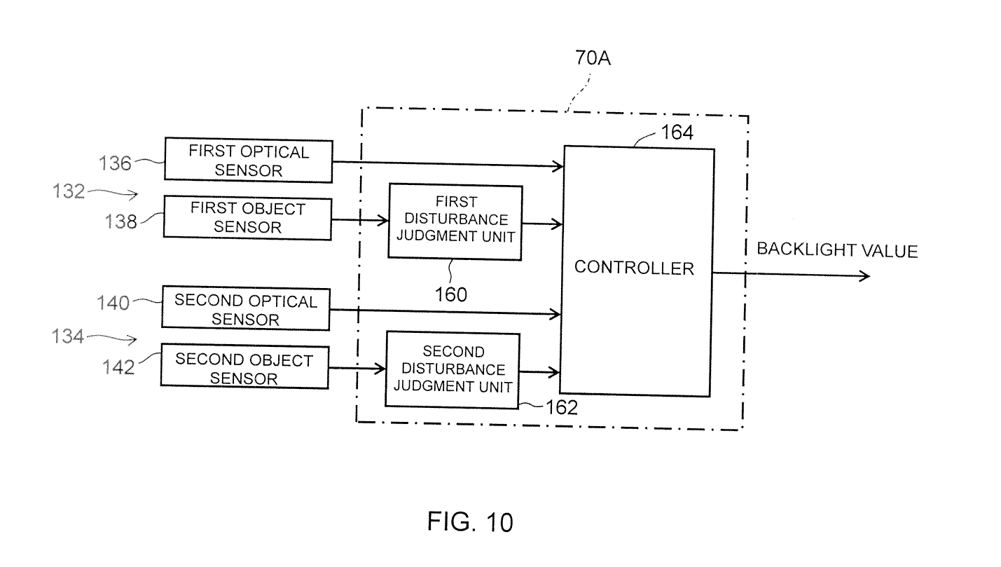

As will be described later with reference to FIG. 10, a controlling unit; that is, a controller, adaptively sets a luminance level of the display screen 30a based on a group of signals which are output from the two sensor pairs 132 and 134. In this case, when an object is detected by the first object sensor 138, a detection value of the first optical sensor 136 is discarded, and similarly, when an object is detected by the second object sensor 142, a detection value of the second optical sensor 140 is discarded. That is, the luminance level is adaptively set based on the light detection values when no object is detected. With such a configuration, an accurate luminance level control while removing or reducing the influence of the disturbances can be realized. In the present embodiment, an LCD is used as the display, and the adjustment of the luminance level is specifically realized by adjustment of a backlight level. However, the method of adjusting the luminance is not limited to such a configuration.

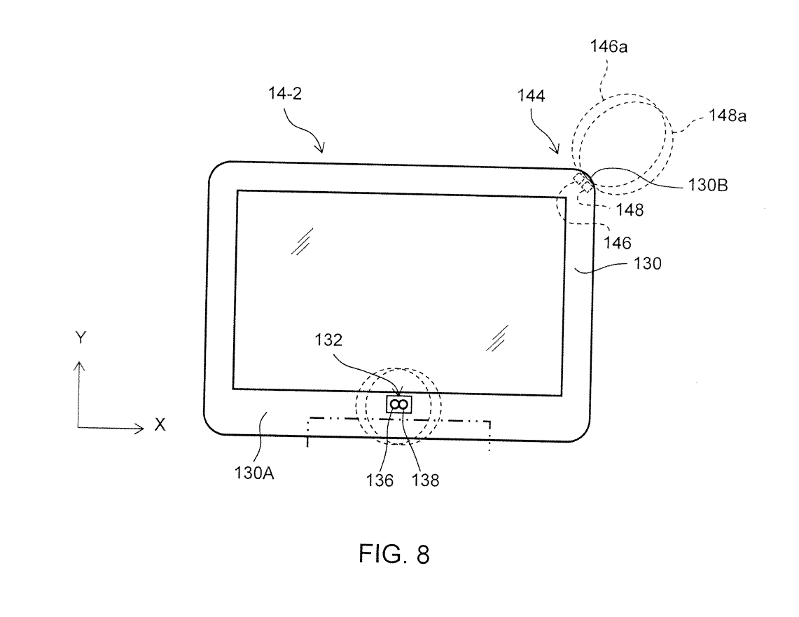

FIG. 8 shows a second example placement of the two sensor pairs. In the second example placement, the first sensor pair 132 is the same as that shown in FIG. 7, and will not be described again.

In the second example placement of FIG. 8, the second sensor pair 144 faces in a upwardly slanted direction. Specifically, the second sensor pair 144 is placed inside a top right corner portion 130B of the frame member 130, and includes a second optical sensor 146 and a second object sensor 148. Directions of central axes of the sensors 146 and 148 are upwardly slanted directions, and more specifically, a 45-degree direction toward the top right of the XY plane. Reference numeral 146a shows a detection field-of-view of the second optical sensor 146, and reference numeral 148a shows a detection field-of-view of the second object sensor 148. The detection fields-of-view 146a and 148a overlap each other in their entire regions.

Therefore, in a BE device 14-2 shown in FIG. 8, two sensor pairs 142 and 144 have different directions. Specifically, the first sensor pair 142 faces to the front, and the second sensor pair 144 faces in an upper right direction. According to such a configuration, a more general ambient light level can be identified without depending on the local strength/weakness of the light. In general, during ultrasound diagnosis, a left side of the frame member 130 is grabbed by the left hand of a right-handed inspector, and a probe is held in the right hand of the inspector or the input operation to the display screen is executed by the right hand. The placement positions of the first sensor pair 142 and the second sensor pair 144 are desirably determined assuming such a configuration, and targeting to reduce the probability that the sensors are hidden by the hands.

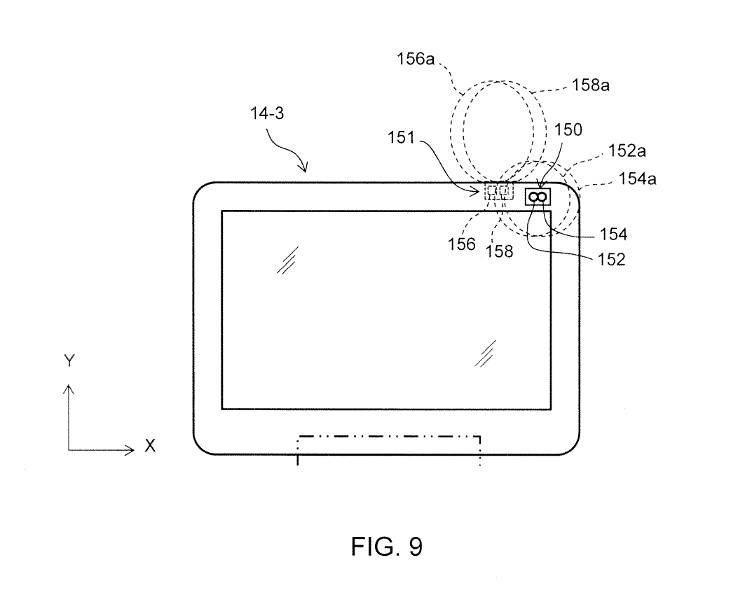

FIG. 9 shows a third example placement of the two sensor pairs. In a BE device 14-3, a first sensor pair 150 and a second sensor pair 152 are placed at or near a top right corner portion of the frame member. In general, in many cases, at a lower part in the display screen, a plurality of operation buttons (icons) are displayed, arranged in the left-and-right direction, and thus the shadow of the hand is more likely to be cast on the lower part of the display screen by respective operations. On the other hand, the shadow of the hand of the inspector is less likely to be cast on the upper part of the frame member, and the shadow of the head of the inspector is also less likely to be cast on the upper part. In consideration of such circumstances, two sensor pairs 150 and 152 are provided at an upper part of the BE device 14-3. The sensor pairs have different directions from each other.

The first sensor pair 150 includes a first optical sensor 152 and a first object sensor 154, and detection fields-of-view thereof are shown by reference numerals 152a and 154a, respectively. The second sensor pair 152 includes a second optical sensor 156 and a second object sensor 158, and detection fields-of-view thereof are shown by reference numerals 156a and 158a, respectively.

As shown in FIG. 9, the first sensor pair 150 is placed facing in the Z direction, that is, to the front, and the second sensor pair 152 is placed to face in the Y direction, that is, upwardly. With such an orthogonal relationship, a more standard ambient light level can be detected. Further, as described above, because the sensor pairs 150 and 152 are provided at the upper part of the BE device 14-3, it is possible to reduce the possibility of the influence of disturbances on the sensor pairs. The sensor structures forming the sensor pairs 150 and 152 are identical to those of the structures shown in FIGS. 7 and 8.

Next, adaptive control of the luminance level will be described with reference to FIG. 10.

In FIG. 10, the first sensor pair 132 includes the first optical sensor 136 and the first object sensor 138 in the illustrated example structure. The second sensor pair 134 includes the second optical sensor 140 and the second object sensor 142. The sensors 132, 134, 140, and 142 correspond to the detectors 84, 86, 88, and 90 shown in FIG. 5. A control unit 70A shown in FIG. 10 corresponds to the CPU 70 shown in FIG. 5.

Functions of the control unit 70A are shown in FIG. 10 as a first disturbance judgment unit 160, a second disturbance judgment unit 162, and a controller 164.

The first disturbance judgment unit 160 judges whether or not there is an occurrence of disturbance, based on an output signal of the first object sensor 138. When an output signal of the first object sensor 138 is greater than or equal to a certain value, the first disturbance judgment unit 160 judges that there is occurrence of the disturbance. The second disturbance judgment unit 162 judges the presence/absence of disturbance based on an output signal of the second object sensor 142, and a judgment principle thereof is identical to the judgment principle of the first disturbance judgment unit 160.

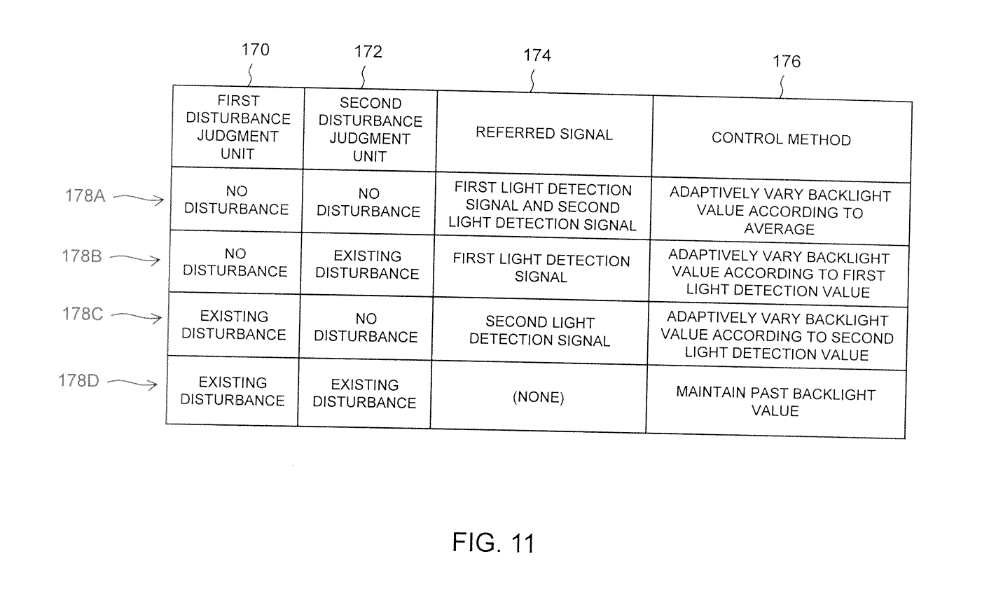

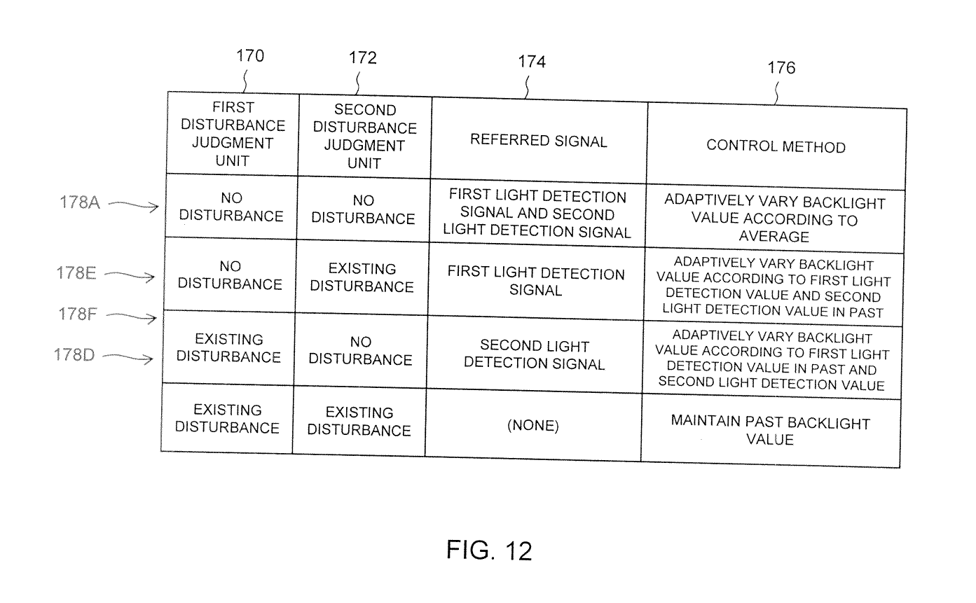

The controller 164 adaptively sets a luminance level of the display, more specifically, the backlight level, based on a detection signal of the first optical sensor 136 and a detection signal of the second optical sensor 140. In the present embodiment, when the first disturbance judgment unit 160 judges a disturbance, that is, when an object is detected, the detection signal of the first optical sensor 136 is discarded, and similarly, when the second disturbance judgment unit 162 judges disturbance, that is, when an object is detected, the detection signal of the second object sensor 142 is discarded. In other words, the controller 164 sets the luminance level based on the detection signals of the first optical sensor and the second optical sensor when there is no disturbance. This process will be described later in detail, with reference to FIGS. 11 and 12. A backlight level of the display is actually set based on a signal showing a backlight value which is output from the controller 164. Alternatively, the luminance signal level may be directly controlled in place of the backlight level, or display conditions such as the contrast and hue may be set.

FIG. 11 shows operation content of the controller shown in FIG. 10 as a table. A row shown by reference numeral 170 shows a judgment result of the first disturbance judgment unit. A row shown by reference numeral 172 shows a judgment result of the second disturbance judgment unit. A row shown by reference numeral 174 shows a signal which is referred to in the control. A row shown by reference numeral 176 shows a control method at the controller. At a state 178A, that is, when the first disturbance judgment unit judges that there is no disturbance and the second disturbance judgment unit also judges that there is no disturbance, the controller variably sets the backlight value according to an average value based on the first light detection signal and the second light detection signal. Specifically, as neither is affected by the disturbance, the light signals for the two optical sensors are both referred to, and the backlight value is determined based on the average value thereof. Alternatively, in this case, a weight may be assigned to each detection value.

In a case of a state 178B; that is, when the first disturbance judgment unit judges that there is no disturbance and the second disturbance judgment unit judges that there is a disturbance, the controller variably sets the backlight value based on the first light detection signal. In other words, the second light detection signal is discarded. This is because, due to the disturbance, the value is not reliable.

In a case of a state 178C, that is, when the first disturbance judgment unit judges that there is a disturbance and the second disturbance judgment unit judges that there is no disturbance, the controller variably sets the backlight value based on the second light detection signal. In other words, the first light detection signal is deduced to be affected by the disturbance, and is discarded.

In a case of a state 178D, that is, when the first disturbance judgment unit judges that there is a disturbance, and the second disturbance judgment unit also judges that there is a disturbance, both light detection signals are not reliable, and the controller executes control to maintain the past backlight value. Alternatively, a configuration may be employed in which, in this case, the current backlight value is calculated based on the light detection signals in a state of no disturbance. In either case, by discarding the light detection signals affected by the disturbance, it becomes possible to avoid a problem whereby an unexpected backlight value is set. With such a configuration, reduction of operability or reduction in visibility can be prevented.

FIG. 12 shows a second example control of the controller. Control identical to that shown in FIG. 11 is assigned the same reference numeral, and will not be described again.

In the example configuration of FIG. 12, control in a state 178E and in a state 178F differs from the control shown in FIG. 11. In a case of the state 178E, that is, when the first disturbance judgment unit judges that there is no disturbance and the second disturbance judgment unit judges that there is a disturbance, in the second example control, the controller variably sets the backlight value based on the first light detection value and a second light detection value in the past. Thus, by also considering the second light detection value in the past which is not affected by the disturbance, it becomes possible to more accurately set the backlight value. Similarly, in a case of the state 178F, that is, when the first disturbance judgment unit judges that there is a disturbance and the second disturbance judgment unit judges that there is no disturbance, the controller determines the backlight value based on a first light detection value in the past and the current second light detection value. Similar to the above, by also considering the first light detection value in the past which is not affected by the disturbance, it becomes possible to more suitably calculate the backlight value. The example controls or calculation methods shown in FIGS. 11 and 12 are merely exemplary.

When gesture input is executed with respect to the BE device, as described above, a motion sensor which detects the motion of the hand may serve as the above-described optical detector. Specifically, during the gesture input, the light detection signal may be discarded, and the display luminance level may be set based on the light detection signal in the case where the gesture input is not executed.

In the present embodiment, the above-described adjustment of the luminance level is executed in the separated state and the docked state. In this case, the control conditions may be set different according to the states. The two sensors of each sensor pair may be placed distanced from each other, but when the two sensors are placed in proximity to each other, it is easy to naturally set the detection fields-of-view to overlap over large portions thereof. In particular, a significant variation occurs in the detection value when a light receiving surface of the optical sensor is covered by a finger or a hand. Thus, in order to accurately detect such a state, desirably, the object sensor is provided in proximity to the optical sensor.

1. An ultrasound diagnostic system comprising:
a front-end device having a transmission and reception circuit; and
a back-end device serving as a tablet terminal, wherein
the front-end device and the back-end device operate in a state selected from a separated state in which the front-end device and the back-end device are separated, and a docked state in which the front-end device and the back-end device are put together,
the back-end device comprises:
a display that displays an ultrasound image which is formed based on a reception signal obtained by transmission and reception of ultrasound, that functions as a touch panel, and on which an input operation to a display screen is executed;
a frame member provided around the display screen of the display;
a first detector pair provided on the frame member facing a front side, the first detector pair including a first optical detector that detects ambient light, and a first object detector that has an object detection field-of-view which is in a relationship of entirely or partially overlapping a light detection field-of-view of the first optical detector;
a second detector pair provided on the frame member facing a direction other than the front side, the second detector pair including a second optical detector that detects the ambient light, and a second object detector that has an object detection field-of-view which is in a relationship of entirely or partially overlapping a light detection field-of-view of the second optical detector,
a disturbance judging unit that judges presence or absence of a disturbance based on an output signal of the first object detector, and that judges presence or absence of a disturbance based on an output signal of the second object detector; and
a control unit that adaptively sets a luminance level of the display based on a first light detection value of the first optical detector when it is judged that there is no disturbance based on the output signal of the first object detector, and a second light detection value of the second optical detector when it is judged that there is no disturbance based on the output signal of the second object detector, in both the separated state and the docked state, wherein
a disturbance judging condition in the disturbance judging unit differs between the separated state and the docked state, and is switched according to the state.

2. The ultrasound diagnostic system according to claim 1, wherein
the control unit sets different conditions for the adaptive setting of the luminance level according to the state selected from the separated state and the docked state.

3. The ultrasound diagnostic system according to claim 1, wherein
the control unit:
refers to the first light detection value and the second light detection value when it is judged that there is no disturbance based on the output signal of the first object detector and that there is no disturbance based on the output signal of the second object detector;
discards the first light detection value and refers to the second light detection value when it is judged that there is a disturbance based on the output signal of the first object detector and that there is no disturbance based on the output signal of the second object detector;
refers to the first light detection value and discards the second light detection value when it is judged that there is no disturbance based on the output signal of the first object detector and that there is a disturbance based on the output signal of the second object detector; and
discards the first light detection value and the second light detection value when it is judged that there is a disturbance based on the output signal of the first object detector and that there is a disturbance based on the output signal of the second object detector the control unit discards the first light detection value when an object is detected by the first object detector.

4.-9. (canceled)
